(19) United States
(12) Patent Application Publication
(10) Pub. No.: US 2024/0047014 A1
(43) Pub. Date: Feb. 8, 2024

(54) FORMULATION GENERATION

(71) Applicants: BASF SE, Ludwigshafen am Rhein (DE); BASF ADVANCED CHEMICALS CO., LTD, Shanghai (CN)

(72) Inventors: Zhe Xiong Yang, Shanghai (CN); Jian Zhang, Shanghai (CN); Zhi Ting Jiang, Shanghai (CN); Johannes Barth, Shanghai (CN)

(21) Appl. No.: 18/268,988

(22) PCT Filed: Dec. 23, 2021
(86) PCT No.: PCT/EP2021/087521

§ 371 Date: Jun. 22, 2023

(30) Foreign Application Priority Data
Dec. 24, 2020 (WO) PCT/CN2020/138955

(51) Int. Cl. G16C 20/10; G16C 20/30

(52) U.S. Cl. CPC G16C 20/10; G16C 20/30; G16C 60/00

(57) ABSTRACT

A computer implemented method for generating synthesis specifications comprising the steps of providing to a computer processor via a communication interface a proposed target application property of the new synthesis specification; providing to the computer processor via the communication interface a data driven model, parametrized based on historical synthesis specifications comprising historical list of components, historical amounts for each of the components and historical target criteria, determining via the computer processor a target synthesis specification based on the data driven model and the target application property providing to an output unit via the communication interface the target synthesis specification, comprising a list of target components and the amount of each of the components.

### TECHNICAL FIELD

The invention relates to a computer implemented method for generating synthesis specifications, a system for generating synthesis specifications and a computer program product for generating synthesis specifications. The invention also relates to use of the generated synthesis specifications for production of chemical products. The invention further relates chemical product manufactured according to a synthesis specification generated by the method for generation of a synthesis specification. The invention further relates to a computer implemented method, a system and a computer program product for synthesis of a chemical product according to the generated synthesis specification

### TECHNICAL BACKGROUND

In chemical industries, finding new chemical compositions for specific target applications properties is a difficult task. Experiments have to be designed based on expert knowledge and exploring the potential parameter space. This quickly becomes very complex and costly due to the large parameter space. More so as the trend goes from commodity chemicals to customer centric solutions, where the customer requests specific target application properties. This requires creation of various chemical compositions and subsequent testing whether they meet the requirements.

### SUMMARY OF THE INVENTION

To address the above mentioned problems the following is proposed;

A computer implemented method for generating a target synthesis specification for a chemical product, in particular a mixture comprising the steps of
providing to a computer processor via a communication interface a target application property;
providing to the computer processor via the communication interface a data driven model, parametrized based on historical synthesis specifications comprising historical chemical components and amounts for each of the components and historical application properties,
determining via the computer processor a target synthesis specification comprising chemical components and amounts for each of the components, based on the data driven model and the target application property
providing to an output unit via the communication interface control data, comprising the target synthesis specification, suitable for synthesizing the chemical product.

The above-mentioned method greatly reduces time and costs in finding new synthesis specifications that provide the target application property. This becomes more relevant as standard chemical products are often commodities and providing customized products becomes more important to distinguish.

According to an aspect use of the method disclosed herein for virtual screening of synthesis specifications is provided.

According to an aspect a synthesis specification determined according to the method disclosed herein is provided.

According to an aspect a chemical product is proposed based on a synthesis specification determined according to the method disclosed herein.

According to an aspect, a computer program or a computer program product or computer readable non-volatile storage medium is disclosed comprising computer readable instructions, which when loaded and executed by a computer processor perform the methods disclosed herein.

According to an aspect a computing apparatus including a processor, a communication interface and a memory storing instructions that, when executed by the processor, configure the apparatus to perform the method disclosed herein.

According to an aspect a computer implemented method of synthesizing a chemical product is proposed comprising the steps of receiving control data generated according to any one of claims 1 to 12 at a control unit, controlling according to the control data, valves associated with vessels containing components of the chemical product, mixing the components in a reactor, providing the chemical product.

In a further aspect a system for producing a chemical product comprising, a control unit configured comprising a processor configured perform the process steps of any one of claim 18 or 19, the system further comprising valves associated with vessels containing component of the chemical product and a reactor in fluid communication with the valves.

A Data driven model may comprise a white box model. “White box model” refers to models based on physico-chemical laws. The physico-chemical laws may be derived from first principles. The physico-chemical laws may comprise one or more of chemical kinetics, conservation laws of mass, momentum and energy, particle population in arbitrary dimension.

The white-box-model may be selected according to the physico-chemical laws that govern the respective problem.

The Data driven model may comprise hybrid models “Hybrid model” refers to a model that comprises white box models, black box models, see e.g. review paper of Von Stoch et al., 2014, Computers & Chemical Engineering, 60, Pages 86 to 101. The trained model may comprise a combination of a white-box-model and a black-box-model.

“Digital representation” may refer to a representation of a material or a mixture of materials e.g. Polymer blends, in a computer readable form. In particular, this may e.g. be a structural formula, a brand name, a CAS number, a synthesis specification, SMILES representation, a representation of polymers in sub-units. INCI name, kinetic model with monomer concentration and process conditions.

“Machine Learning” may refer to computer algorithms that improve through experience, Machine Learning algorithms build a model based on sample data, often described as training data.

“Communication interface” may refer to a software and/or hardware interface for establishing communication such as transfer or exchange or signals or data. Software interfaces may be e. g. function calls, APIs. Communication interfaces may comprise transceivers and/or receivers. The communication may either be wired, or it may be wireless. Communication interface may be based on or it supports one or more communication protocols. The communication protocol may a wireless protocol, for example: short distance communication protocol such as Bluetooth®, or WiFi, or long communication protocol such as cellular or mobile network, for example, second-generation cellular network or (“2G”), 3G, 4G, Long-Term Evolution (“LTE”), or 5G. Alternatively, or in addition, the communication interface may even be based on a proprietary short distance or long distance protocol. The communication interface may support any one or more standards and/or proprietary protocols.

“Computer processor” may refer to an arbitrary logic circuitry configured for performing basic operations of a computer or system, and/or, generally, to a device which is configured for performing calculations or logic operations. In particular, the processing means, or computer processor may be configured for processing basic instructions that drive the computer or system. As an example, the processing means or computer processor may comprise at least one arithmetic logic unit (“ALU”), at least one floating-point unit (“FPU)”, such as a math coprocessor or a numeric coprocessor, a plurality of registers, specifically registers configured for supplying operands to the ALU and storing results of operations, and a memory, such as an L1 and L2 cache memory. In particular, the processing means, or computer processor may be a multicore processor. Specifically, the processing means, or computer processor may be or may comprise a Central Processing Unit (“CPU”). The processing means or computer processor may be a (“CISC”) Complex Instruction Set Computing microprocessor, Reduced Instruction Set Computing (“RISC”) microprocessor, Very Long Instruction Word (“VLIW”) microprocessor, or a processor implementing other instruction sets or processors implementing a combination of instruction sets. The processing means may also be one or more special-purpose processing devices such as an Application-Specific Integrated Circuit (“ASIC”), a Field Programmable Gate Array (“FPGA”), a Complex Programmable Logic Device (“CPLD”), a Digital Signal Processor (“DSP”), a network processor, or the like. The methods, systems and devices described herein may be implemented as software in a DSP, in a micro-controller, or in any other side-processor or as hardware circuit within an ASIC, CPLD, or FPGA. It is to be understood that the term processing means or processor may also refer to one or more processing devices, such as a distributed system of processing devices located across multiple computer systems (e.g., cloud computing), and is not limited to a single device unless otherwise specified.

Synthesis specification may refer to instructions for synthesizing a chemical product. In particular comprising the components, the amounts of each of the components and may also comprise instructions on conditions, such as temperature, or mixing instructions.

Amount of a component may refer to the distribution of individual components in a chemical product. It may refer to the concentration of each component (as molar fraction, volume fraction, mass fraction, molality, molarity or normality or mixing ratio). Amount may also refer to absolute values (e.g. volumes, masses)

In an aspect the data driven model may be a generative model. Using a generative model has the advantage that with relatively low computational power new synthesis specifications can be determined that will meet the target application property. This may greatly reduce effort in finding new synthesis specifications meeting a target application property. Therefore, the method may be applied for screening. Generative models may refer to data driven models that can generate new data instance. Suitable generative models may be Bayesian net, Markov chain, autoregressive model, latent variable model, implicit density model, RNN, CNN, VAE, GAN.

In an aspect the data driven model is parametrized based on a sequence of the amounts for each of the components. The term sequence may refer to any ordering, where the proportion of the components is sorted in a descending or ascending order in form of an ordered list. The information is often already inherent in various synthesis specifications, e. g. in personal care products, such as nutrition, pharmaceuticals, cosmetics, home care the synthesis specifications are ordered in a descendent order of the amount of each of the components. The sequence of the amount of each of the components may not comprise absolute values or concentrations. In an example the sequence of the amount of each of the components may comprise a relative order such as more than or less than a previous component in the ordered list.

When a sequence of the amount of each of the components is not readily available, the historical synthesis specifications may be preprocessed such that a sequence of the amount of each of the components is generated. An example could be a synthesis specification, where the components and the concentrations are known, but no sequential order is presented. An optional preprocessing step may then comprise sorting the components in a descending or ascending order and thereby generating a sequence of the amount of each of the components.

By that additional information is provided to the model. This may allow using a reduced data set. It can dramatically decrease the cost of building the data-driven model if that additional information is used. This also means that each component also depends on the previous component in the ordered list. In addition, this may allow treating the sequence of the proportion of components as time series.

In an aspect the data-driven model may comprises a stop token component. The stop token component may indicate the end of the sequence. The stop token component is a virtual component, that has no other purpose than indicating the end of the synthesis specification.

In an aspect the data driven model may comprise a start token component. A start token component provides a standardized way of initializing the data driven model.

In an aspect, the data driven model-model comprises components contained in the historic synthesis specifications used for training. The components may be provided in form of a list. This list may be complete list containing all components, in an alternative the list may be a reduced list of the components contained in the synthesis specifications used for training. A complete list may advantageous if the goal is a complete synthesis specification, a reduced list may be beneficial if synthesis specifications may contain components that have no relevance for the target application property. An example could be where the target application property is a sun-screen personal care product and fragrance do not provide a contribution to the sun protection. In that case, the target synthesis specification may be determined from a data-driven model comprising a reduced list of components, wherein the list only contains components relevant for the target application property. Other examples where reduced lists may be beneficial could be coatings and/or paints, where the target application property is a certain physical property and the color may be added later for example in form of a pigment.

In an aspect step of determining via the computer processor a target synthesis specification based on the data driven model and the target application property may comprise the step of repeatedly sampling components from the data driven model. In this case the data driven model may comprise a probability distribution for each component.

Sampling from a data driven model comprising a probability distribution for each component has the advantage that a sequence of the components of the target synthesis specification will be similar to the sequence of components of known synthesis specifications which form the historical synthesis specifications in the data driven model.

The aspect of providing a data driven model comprising a stop token component and the step of reportingly sampling from a data driven model comprising a probability distribution for each component has a synergetic effect. The sampling process may be repeated until the target synthesis specification comprises the stop token component. Providing in the data driven model the stop token component indicating the end of the provides a definition, when the synthesis specification is complete. When the sequence of components is sorted in descending order than the method could potentially provide very long lists containing neglectable amounts of components, e. g. a large number of ingredients, with negligible amounts. This can be prevented by providing an end of sequence indicator that defines a limit to the list of components. Besides, it allows the model terminated the generating process randomly, which makes the model generate component list with different length automatically, this adds another benefit.

In an aspect the data driven model may comprise a recurrent neural network. Recurrent neural networks are in particular useful for time series. In this invention the concepts for time series is transferred to synthesis specifications.

In an aspect the target application property may be provided in a vector representation of the target application property. The advantage of providing the target application property as a vector representation reduces the dimension of the target application property and therefore reduces complexity of the model, thereby reducing computational costs. Furthermore, the robustness of the data driven model is increased, in particular, when deep neural networks are used as these are very vulnerable with the parameter dynamics during training. This results in a more reliable generation of a synthesis specification.

The vector representation may also be used to determine similarities between target criteria. In case of personal care products, a body lotion may be similar to a sunscreen product both of are skin care products the difference may lie in the sun protection provided by the sun screening effect.

In an aspect the components may be provided in a vector representation.

The advantage of providing components as a vector representation reduces the dimension of the target application property and therefore reduces complexity of the model, thereby reducing computational costs. Furthermore, the robustness of the data driven model is increased, in particular, when deep neural networks are used deep neural network are used as these are very vulnerable with the parameter dynamics during training.

In an aspect the method of generating synthesis specifications may further comprise a validating step. In an aspect the validation process may comprise assessing whether additional requirements are met. These additional requirements may comprise that the target synthesis specification differs from the historical synthesis specifications.

The validating step may comprise comparing the target synthesis specification to historical synthesis specifications and only providing the target synthesis specification when the target synthesis specification differs from the historical synthesis specifications. The additional requirements may comprise a phase stability parameter, e.g. a target shelf life and the validating step may comprise providing the target stability parameter to a system for predicting a target shelf life, receiving a predicted phase stability parameter and comparing the predicted phase stability parameter to the target phase stability parameter.

In an aspect the target application property may be provided in form of an application identifier, the application identifier may be an easily interpretable form, such as for example the intended application of the synthesis specification or any other target application property. The corresponding vector representation of the target application property may then be derived from the application identifier, for example by selecting from a database. This allows to provide the target application property in an easily understandable way. For the purpose of generating a synthesis specification a vector representation is useful. Unfortunately this is not a very intuitive representation, therefore, the step of providing to the computer processor via the communication interface the target application property in form of an application identifier may improve usability.

Additionally or alternatively the validating step may comprise a step of assessing whether at least one of the components is an active component for the target application property. Turning again to the example of a sun screen, active components may be organic chemical compounds that absorb UV light, inorganic particles that reflect scatter and adsorb UV light or organic particles that mostly absorb UV light like organic chemical compounds, but contain multiple chromophores that reflect and scatter a fraction of light like inorganic particles. Examples of active components are p-Aminobenzoic acid, Padimate O, Phenylbenzimidazole sulfonic acid, Cinoxate, Dioxybenzone, Oxybenzone, Homosalate, Menthyl anthranilate, Octocrylene, Octyl methoxycinnamate, Octyl salicylate, Sulisobenzone, Trolamine salicylate, Avobenzone, Ecamsule, Titanium dioxide, Zinc oxide, 4-Methylbenzylidene camphor, Parsol Max, Tinosorb M, Parsol Shield, Tinosorb S, Tinosorb A2B, Neo Heliopan AP, Mexoryl XL, Benzophenone-9, Uvinul T 150, Uvinul A Plus, Uvasorb HEB, Parsol SLX, Amiloxate.

Any disclosure and embodiments described herein relate to the methods, the systems, the treatment devices, the computer program element lined out above and vice versa.

Advantageously, the benefits provided by any of the embodiments and examples equally apply to all other embodiments and examples and vice versa.

As used herein “determining” also includes “initiating or causing to determine”, “generating” also includes “initiating or causing to generate” and “providing” also includes “initiating or causing to determine, generate, select, send or receive”. “Initiating or causing to perform an action” includes any processing signal that triggers a computing device to perform the respective action.

### BRIEF DESCRIPTION OF THE DRAWINGS

To easily identify the discussion of any particular element or act, the most significant digit or digits in a reference number refer to the figure number in which that element is first introduced.

FIG. 1 illustrates a routine 100 for generating synthesis specifications in accordance with one embodiment.

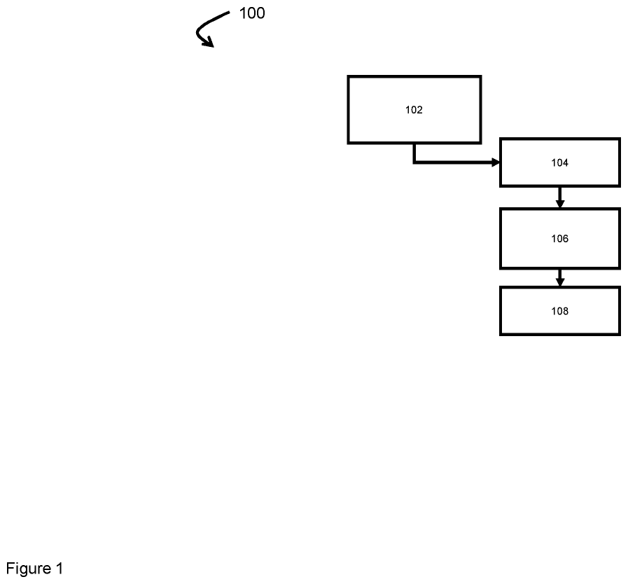

FIG. 2 illustrates an aspect of the subject matter in accordance with one embodiment.

FIG. 3 illustrates an aspect of a system for generating a synthesis specification.

FIG. 4 illustrates an aspect of the subject matter in accordance with one embodiment.

FIG. 5 illustrates a workflow for producing a chemical product.

FIG. 6 illustrates a system for producing a chemical product.

### DETAILED DESCRIPTION

Non limiting examples of the implementation of the invention are provided with respect to the figure description below. For illustration purposes various optional features are combined in the provided examples. It is apparent for those skilled in the art that not all features are necessary for executing the invention.

In block 102, routine 100 provides to a computer processor via a communication interface a proposed target application property, wherein in this example the target application property is provided in a vector representation. In block 104, routine 100 provides to the computer processor via the communication interface a data driven model, parametrized based on historical synthesis specifications comprising historical list of components, historical amounts for each of the components and historical target criteria, wherein the date driven model comprises a generative model, is parametrized based on a sequence of the amounts for each of the components, comprises a stop token component, comprises a start token component, comprises a list of components contained in the historic synthesis specifications, is parametrized based on a vector representation of historical target criteria and a vector representation of the components in the list of components, comprises a recurrent neural network. In block 106, routine 100 determines via the computer processor a target synthesis specification based on the data driven model and the target application property, wherein the step of determining further comprises repeatedly sampling components from the data driven model. In block 108, routine 100 provides to an output unit via the communication interface the target synthesis specification, comprising a list of target components and the amount of each of the components.

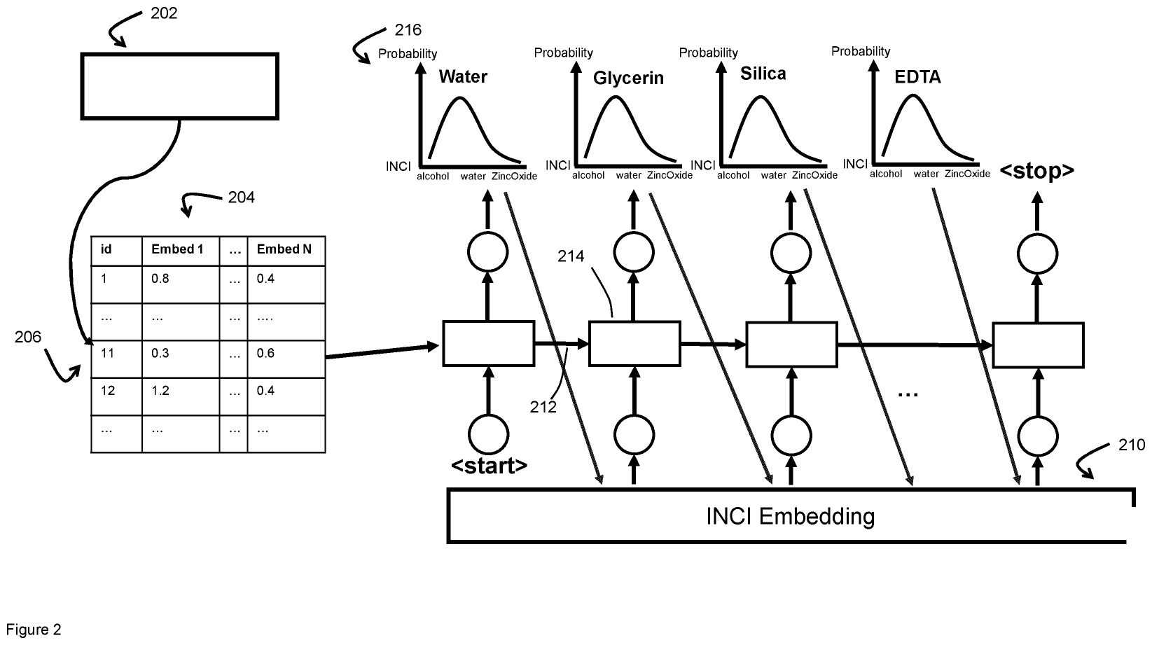

In FIG. 2 a workflow of a recurrent network for executing the method according to the invention is shown. A target application property 202 is provided in form of an application identifier. In this example the application identifier may be “sunscreen” as the application of the new synthesis specification is intended to be a sun protection personal care product. The corresponding vector representation of the target application property 206 is then selected from a database 204. The vector representation of the target application property 206 may be one hot vector, however, more complex vector representations may be chosen. This allows to provide the target application property in an easily understandable way. For the purpose of generating a synthesis specification vector representation of the target application property 206 vector representation is useful. The vector representation is then used to initialize the data driven model, which in this example is a recurrent neural network. The data driven model further comprises list of components 210 in this example this may be in the form of a component database 210. The list of components may comprise a vector representation of the components in the list of components. The vector representation of the components in the list of components may be one hot vector for each of the components, however, more complex vector representations may be chosen. A start token component is selected as an input x₀ into the recurrent neural network. A hidden state h₀ provides a probability distribution of the components. This represents the probability that an component will be at that position of the sequence of the new synthesis specification. In a sampling step the next component x₁ is sampled from the probability distribution. In this example, the next component would be water. The output 212 of the recurrent neural network contains information on the previously sampled component x₀, and the vector representations of the target application property 206. The output 212 is then fed as input 214 into the recurrent neural network. Next, the component x₁ is provided as an input into the recurrent neural network. A hidden state h₁ provides a probability distribution of the components. This represents the probability that an component will be at that position of the sequence of the new synthesis specification. An exemplary probability distribution is denoted 216. In a sampling step the next component x₂ is sampled from the probability distribution. In this example, the next component would be glycerin. The output 212 of the recurrent neural network contains information on previously sampled the components x₀ and x₁, and the vector representations of the target application property 206. The output 212 is then fed as input 214 into the recurrent neural network. This sampling step is repeated until the stop token component xₙ is sampled. The new synthesis specification comprises the components x₁, . . . , xₙ₋₁ as a sequence. The sequence may then be provided via the communication interface.

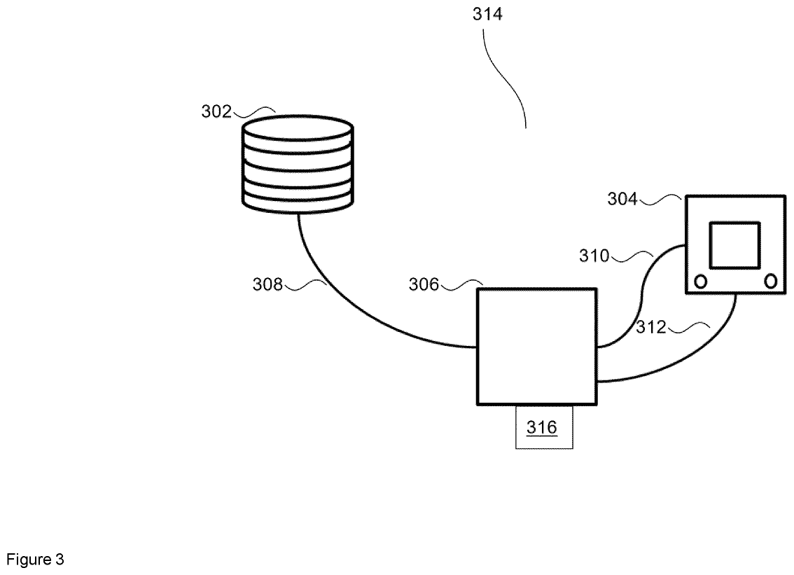

FIG. 3 shows an example of a computing apparatus 314 comprising: a computer processor 306; a communication interface 308,310, 312 a memory 316 storing instructions that, when executed by the processor, configure the apparatus to perform the steps of provide to the computer processor via the communication interface a target application property; provide to the computer processor via the communication interface a data driven model parametrized based on parametrized based on historical synthesis specifications comprising historical list of components, historical amounts for each of the components and historical target criteria, determine via the computer processor a target synthesis specification based on the data driven model and the target application property and provide to an output device via the communication interface the target synthesis specification, comprising a list of target components and the amount of each of the components.

In this example the computer apparatus further comprises in input/output device 304. In this example the data driven model is stored in a data base 302. The data base 302 is connected to the computer processor via the communication interface 308. In this example input/output device 304 is used to provide a target application property via communication interface 310. In this example the target application property is provided in the form of an application identifier. The application identifier in that case may be “sunscreen. The computer processor 306 then retrieves from the data base 302 vector representation of the target application property. The vector representation of the target application property is then provided to the computer processor 306. The data driven model is provided to the computer processor 306 via the communication interface 308. With the computer processor 306 a target synthesis specification determined based on based on the data driven model and the target application property. The target synthesis specification is provided to the input/output device 304 via communication interface 312. In another example the phase stability parameter may be provided to the data base 302 via communication interface 308.

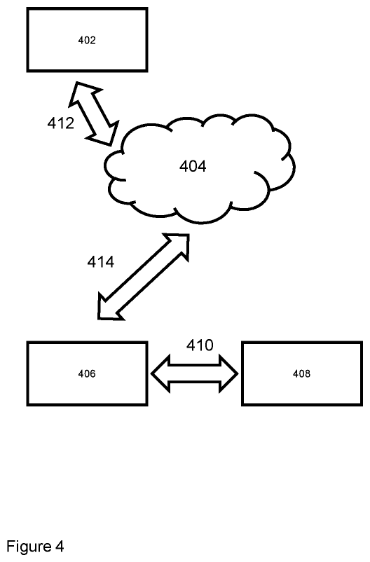

Turning to FIG. 4, there is shown an Internet-based system for generating a synthesis specification. The system 400 comprises a server 402 which can be accessed via a network 404, such as the Internet, by one or more client devices 406.1 to 406.n. The server may be an HTTP server and is accessed via conventional Internet web-based technology. The client devices 406 may be computer terminals accessible by a user and may be customized devices, such as data entry kiosks, or general-purpose devices, such as a personal computer. A printer 408 can be connected to a client device 406. The internet-based system is in particular useful, if a service is provided to customers or in a larger company setup. A client may be used to provide the target application property to the computer processor of the server.

In an aspect the client may generate a request to initiate the generation of a synthesis specification based on a target application property, wherein the client device is configured to provide the target application property to the server device.

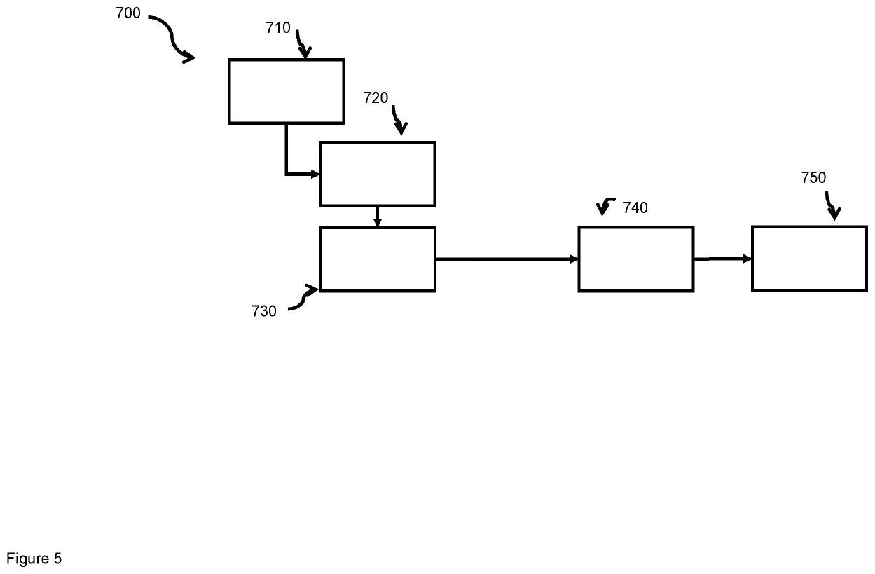

FIG. 5 depicts an exemplary workflow 700 for producing a chemical product. At step 710 control data comprising synthesis specifications of a chemical product are provided. The control data comprise information associated with the components and the amount of each of the components. At step 720 valves associated with vessels containing the components are controlled according to the control data such that in a reactor the components are provided in the amounts a specified in the synthesis specification. At optional step 730 a mixer in the reactor may be controlled to ensure a good mixture of the components. At optional step 740 heaters may be controlled for heating the components according to the synthesis specification. At step 750 the chemical product is provided. This may be performed by opening an exit valve at the reactor.

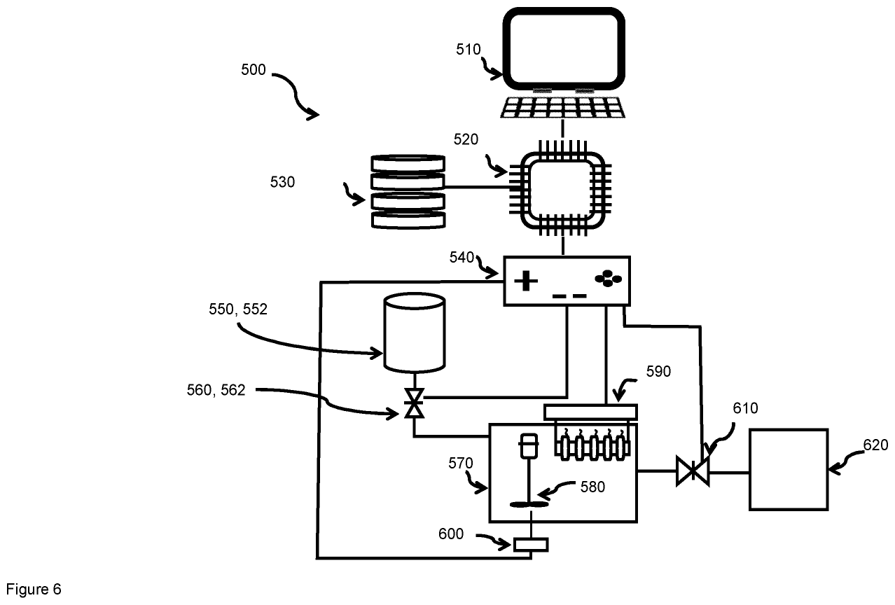

FIG. 6 shows a system 500 for producing a chemical product based on a synthesis specification generated according to the invention. In this example the system comprises a user interface 510 and a processor 520, associated with a control unit 540, the control unit is configured for receiving control data generated according to the invention. In this example the control data are provided from a data base 530, in other examples, the control data may be provided from a server. Vessels 550, 552 each contain a component of the chemical product. In general more than two vessels are present. For illustration purposes the example only shows two vessels. Valves 560, 562 are associated with vessels 550, 552. Valves 550 and 552 may be controlled to dose appropriate amounts of each component in reactor 570, according to the synthesis specification. A motor 600 of a mixer 580 may also be controlled by the control unit according to the synthesis specification. An optional heater 590 may also be controlled according to the synthesis specification. Finally, an exit valve 610 in fluid communication with the reactor may be controlled by the control unit to provide the chemical product to a container or test system 620.

1. A computer implemented method for generating a target synthesis specification for a chemical product, in particular a mixture comprising:
providing to a computer processor via a communication interface a target application property;
providing to the computer processor via the communication interface a data driven model, parametrized based on historical synthesis specifications comprising historical chemical components and amounts for each of the components and historical application properties,
determining via the computer processor a target synthesis specification comprising chemical components and amounts for each of the components, based on the data driven model and the target application property
providing to an output unit via the communication interface control data, comprising the target synthesis specification, suitable for synthesizing the chemical product.

2. The computer implemented method of claim 1, wherein providing the data driven model comprises providing to the processing device a generative model.

3. The computer implemented method of claim 1, wherein the data driven model is parametrized based on a sequence of the amounts for each of the components.

4. The computer implemented method of claim 1, wherein the data driven model comprises a stop token component and/or a start token component.

5. The computer implemented method of claim 1, wherein the data driven model comprises a list of components contained in the historic synthesis specifications.

6. The computer implemented method of claim 1, wherein the target application property is provided in a vector representation.

7. The computer implemented method of claim 1, wherein the data driven model is parametrized based on a vector representation of historical target criteria and a vector representation of the components in the list of components.

8. The computer implemented method of claim 1, wherein determining via the computer processor a target synthesis specification based on the data driven model and the target application property comprises:
repeatedly sampling components from the data driven model.

9. The computer implemented method of claim 1, wherein the data driven model comprises a recurrent neural network.

10. The computer implemented method of claim 1, further comprising the step of validating the synthesis specification.

11. The computer implemented method according to claim 1, wherein the target application property is an application in a field selected from the group of personal care, coatings, polyurethane A-component, adhesives, home care, agricultural chemical synthesis specifications, nutrition.

12. The computer implemented method of claim 1, wherein providing the target application property comprises providing the target application property via a client device and receiving the control data at the client device.

13. A computing apparatus including a processor, a communication interface and a memory storing instructions that, when executed by the processor, configure the apparatus to perform the method of claim 1.

14. A non-transitory computer-readable storage medium including instructions that, when processed by a computer, configure the computer to perform the method of claim 1.

15. (canceled)

16. (canceled)

17. A chemical product, based on a synthesis specification determined according to the method of claim 1.

18. A computer implemented method of synthesizing a chemical product comprising:
receiving control data generated according to claim 1 at a control unit,
controlling according to the control data, valves associated with vessels containing components of the chemical product,
mixing the components in a reactor, in fluid communication with the valves
providing the chemical product.

19. The method of claim 18, wherein providing the chemical product further comprising providing the chemical product to a test system via an exit valve.

20. A system for producing a chemical product comprising, a control unit configured comprising a processor configured to perform the process steps of claim 18, the system further comprising valves associated with vessels containing component of the chemical product and a reactor in fluid communication with the valves.
